(19) United States
(12) Patent Application Publication
(10) Pub. No.: US 2005/0042752 A1
(43) Pub. Date: Feb. 24, 2005

(54) Methods of inducing gene expression

(76) Inventors: Patrice Crete, Marseille (FR); Ulrich Gerhard Klahre, Pfeffingen (CH); Frederick Meins, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/490,489

(22) PCT Filed: Sep. 27, 2002
(86) PCT No.: PCT/EP02/10889

(30) Foreign Application Priority Data
Sep. 28, 2001 (GB) 0123401.2

(51) Int. Cl. C12N 15/85

(52) U.S. Cl. 435/455

(57) ABSTRACT

The present invention relates to methods of regulating gene expression in eukaryotic cells, in particular providing stable and persistent expression that can undergo systemic spreading in plants.

The present invention relates to the field of molecular biology, in particular to the regulation of gene expression by gene silencing. The technology has wide applications including developing therapeutic methods for treating diseases and expressing desired products in agricultural crops.

### BACKGROUND OF THE INVENTION

Posttranscriptional gene silencing (PTGS) is an epigenetic form of mRNA degradation important in the defense of plants against virus infection and widely used as a tool for inactivating gene expression (Kooter et al., 1999; Fire, 1999; Vance and Vaucheret, 2001). PTGS can arise spontaneously in transgenic plants and can be induced systemically by the local introduction of additional gene copies. Earlier studies have established that transgenes likely to generate double-stranded RNAs (dsRNAs) efficiently promote PTGS in plants (Hamilton et al., 1998; Waterhouse et al., 1998; Smith et al., 2000; Chuang and Meyerowitz, 2000; Sijen et al., 2001); and, that dsRNAs delivered into cereal cells can block gene expression, which is limited to the target cell (Schweizer et al., 2000).

First discovered in plants, it is now recognized that PTGS or the closely related phenomenon, RNA interference (RNAi), occurs in many organisms including Neurospora crassa, Trypanosoma brucei, Caenorhabditis elegans, Drosophila, mouse and humans (Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498; Fire, 1999; Fagard and Vaucheret, 2000a; Kooter et al., 1999; Meins, 2000; Sharp, 2001). The identification of homologous genes essential for both RNAi and PTGS in Arabidopsis, N. crassa, and C. elegans suggests a common, highly conserved, underlying mechanism (Catalanotto et al., 2000; Cogoni and Macino, 2000; Fagard et al., 2000).

PTGS in plants and animals is associated with production of small sense and antisense RNAs (smRNAs) representing regions of the silenced genes (Hamilton and Baulcombe, 1999; Zamore et al., 2000; Hutvagner et al., 2000). Double-stranded smRNAs target RNAs for degradation in Drosophila-embryo extracts (Elbashir et al., 2001) and can trigger PTGS when introduced into cultured mammalian cells (Elbashir et al., 2001). Similalry, RNAi in animals is mediated by double-stranded RNAs (dsRNAs) that undergo endonucleolytic cleavage to generate small sense and antisense RNAs (smRNA) 21 to 23 nucleotides in length, which then promote RNA degradation (Tuschl et al., 1999; Zamore et al., 2000; Elbashir et al., 2001). Inhibition of the expression of a nucleotide sequence mediated by dsRNA interference is described in WO 99/32619, WO 99/53050 or WO 99/61631.

In the last few years, advances in nucleic acid chemistry and gene transfer have inspired new approaches to engineer specific inhibition of gene expression. However, specific induction of expression is also a desirable objective, which has up until now generally been achieved with varying degrees of effectiveness by administering costly or toxic chemical compounds. Jepson et al. (1998) reviews various chemical-inducible gene expression systems for plants and further describe the desirable properties of an inducible expression system. Repression of gene expression using the tet repressor-operator system and its use for specific induction of gene expression with tetracycline has also been described previously (Gatz et al., 1991). Nevertheless, there is still a need for methods allowing one to induce gene expression effectively and predictably, in a cost-effective manner, and this invention meets that need.

### RELEVANT LITERATURE

Anandalakshmi, R., Marathe, R., Ge, X., Herr, J. M. Jr., Mau, C., Mallory, A., Pruss, G., Bowman, L., and Vance, V. B. (2000). A calmodulin related protein that suppresses posttranscriptional gene silencing in plants. Science 290, 142-144.
Catalanotto, C., Azzalin, G., Macino, G., and Cogoni, C. (2000). Gene silencing in worms and fungi. Nature 404, 245.
Chuang, C. F. and Meyerowitz, E. M. (2000). Specific and heritable genetic interference by double-stranded RNA in Arabidopsis thaliana. Proc. Natl. Acad. Sci. USA 97, 4985-4990.
Cogoni, C. and Macino, G. (2000). Post-transcriptional gene silencing across kingdoms. Curr. Opin. Genet. Dev. 10, 638-643.
Dalmay, T., Hamilton, A., Rudd, S., Angell, S., and Baulcombe, D. C. (2000). An RNA-dependent RNA polymerase gene in Arabidopsis is required for posttranscriptional gene silencing mediated by a transgene but not by a virus. Cell 101, 543-553.
Di Serio, F., Schöb, H., Iglesias, A., Tarina, C., Bouldoires, E., and Meins, F. Jr. (2001). Sense- and antisense-mediated gene silencing in tobacco is inhibited by the same viral suppressors and is associated with accumulation of small RNAs. Proc. Natl. Acad. Sci. USA 98, 6506-6510.
Duckett, C. M., Oparka, K. J., Prior, D. A. M., Dolan, L., and Roberts, K. (1994). Dye-coupling in the root epidermis of Arabidopsis is progressively reduced during development. Development 120, 3247-3255.
Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.
Elbashir, S. M., Lendeckel, W., and Tuschl, T. (2001). RNA interference is mediated by 21 and 22 nt RNAs. Genes Devel. 15, 188-200.
Fagard, M., Boutet, S., Morel, J.-B., Bellini, C., and Vaucheret, H. (2000). AGO1, QDE-2, and RDE-1 are related proteins required for post-transcriptional gene silencing in plants, quelling in fungi, and RNA interference in animals. Proc. Natl. Acad. Sci. USA 97, 11650-11654.
Fagard, M. and Vaucheret, H. (2000a). (Trans)gene silencing in plants: How many mechanisms? Annu. Rev. Plant Physiol. Plant Mol. Biol. 51, 168-221.
Fagard, M. and Vaucheret, H. (2000b). Systemic silencing signal(s). Plant Mol. Biol. 43, 285-293.
Fire, A. (1999). RNA-triggered gene silencing. Trends in Genet. 15, 358-363.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Gatz, C., Kaiser, A., and Wendenburg, R. (1991). Regulation of a modified CaMV 35S promoter by the Tn10-encoded Tet repressor in transgenic tobacco.
Gatz, C., Frohberg, C., and Wendenburg, R. (1992). Stringent repression and homogeneous de-repression by tetracycline of a modified CaMV 35S promoter in intact transgenic tobacco plants. Plant J. 2, 397-404.
Goodwin, P. B., Shepard, V., and Erwee, M. G. (1990). Compartmentation of fluorescent tracers injected into the epidermal cells of Egeria densa leaves. Planta 181, 129-136.
Grishok, A., Pasquinelli, A. E., Conte, D., Li, N., Parrish, S., Ha, I., Baille, D. L., Fire, A., Ruvkun, G., and Mello, C. C. (2001). Genes and Mechanisms Related to RNA Interference Regulate Expression of the Small temporal RNAs that Control C. elegans Developmental Timing. Cell 106, 23-34.
Hamilton, A. J. and Baulcombe, D. C. (1999). A species of small antisense RNA in posttranscriptional gene silencing in plants. Science 286, 950-952.
Hamilton, A. J., Brown, S., Yuanhai, H., Ishizuka, M., Lowe, A., Solis, A.-G. A., and Grierson, D. (1998). A transgene with repeated DNA causes high frequency, post-transcriptional suppression of ACC-oxidase gene expression in tomato. Plant J. 15, 737-746.
Haseloff, J., Siemering, K. R., Prasher, D. C., and Hodge, S. (1997). Removal of a cryptic intron and subcellular localization of green fluorescent protein are required to mark transgenic Arabidopsis plants brightly. Proc. Natl. Acad. Sci. USA 94, 2122-2127.
Horsch, R., Fry, J., Hoffman, N., Neidermeyer, J., Rogers, S., and Fraley, R. (1988). Leaf disc transformation. In Plant molecular biology manual, A5, S. Gelvin and R. Schilperoort, eds (Dordrecht: Kluwer Academic), pp. 23-50.
Hutvagner, G., Mlynarova, L., and Nap, J. P. (2000). Detailed characterization of the posttranscriptional gene-silencing related small RNA in a GUS gene-silenced tobacco. RNA 6, 1445-1454.
Iglesias, V. A., Gisel, A., Bilang, R., Leduc, N., Potrykus, I., and Sautter, C. (1994). Transient expression of visible marker genes in meristem cells of wheat embryos after ballistic micro-targeting. Planta 192, 84-91.
Jepson, I., Martinez, A., and Sweetman, J. P. (1998) Chemical-inducible Gene Expression Systems for Plants—a Review. Pestic. Sci. 54, 360-367.
Jones, A. M., Im, K.-H., Savka, M. A., Wu, M.-J., DeWitt, N. G., Shillito, R., and Binns, A. N. (1998). Auxin-dependent cell expansion mediated by overexpressed auxin-binding protein 1. Science 282, 1114-1117.
Klahre, U. and Chua, N.-H. (1999). The Arabidopsis ACTIN-RELATED PROTEIN 2 (atARP2) promoter directs expression in xylem precursor cells and pollen. Plant Mol. Biol. 41, 65-73.
Kooter, J. M., Matzke, M. A., and Meyer, P. (1999). Listening to the silent genes: transgene silencing, gene regulation and pathogen control. Trends Plant Sci. 4, 340-347.
Lang, A. (1965). Progressiveness and contagiousness in plant differentiation and development. Encycl. Plant Physiol. 15(1), 409-423.
Linsmaier, B. and Skoog, F. (1965). Organic growth factor requirements of tobacco tissue culture. Physiol. Plant. 18,100-127.
Mallory, A. C., Ely, L., Smith, T. H., Marathe, R., Anandalakshmi, R., Fagard, M., Vaucheret, H., Pruss, G., Bowman, L., and Vance, V. B. (2001). HC-Pro suppression of transgene silencing eliminates the small RNAs but not transgene methylation or the mobile signal. Plant Cell 13, 571-583.
Meins, F. Jr. (2000). RNA degradation and models for posttranscriptional gene silencing. Plant Mol. Biol. 43, 261-273.
Meyer, A. D., Aebi, R., and Meins, F. Jr. (1997). Tobacco plants carrying a tms locus of Ti plasmid origin and the HI-1 allele are tumor prone. Differentiation 61, 213-221.
Mourrain, P., Beclin, C., Elmayan, T., Feuerbach, F., Godon, C., Morel, J. B., Jouette, D., Lacombe, A.-M., Nikic, S., Picault, N., Remoue, K., Sanial, M., Vo, T. A., and Vaucheret, H. (2000). Arabidopsis SGS2 and SGS3 genes are required for posttranscriptional gene silencing and natural virus resistance. Cell 101, 533-542.
Oparka, K. J., Roberts, A. G., Boevink, P., Santa Cruz, S., Pradel, K. S., Imlau, A., Kotlizky, G., Sauer, N., and Epel, B. (1999). Simple, not branched, plasmodesmata allow the nonspecific trafficking of proteins in developing tobacco leaves. Cell 97, 743-754.
Palauqui, J., Elmayan, T., Pollien, J., and Vaucheret, H. (1997). Systemic acquired silencing: transgene-specific post-transcriptional silencing is transmitted by grafting from silenced stocks to non-silenced scions. EMBO J. 16, 4738-4745.
Palauqui, J. C. and Balzergue, S. (1999). Activation of systemic acquired silencing by localized introduction of DNA. Curr. Biol. 9, 59-66.
Parrish, S., Fleenor, J., Xu, S., Mello, C., and Fire, A. (2000). Functional anatomy of a dsRNA trigger. Differential requirement for the two trigger strands in RNA interference. Mol. Cell 6, 1077-1087.
Pieterse, C. M. J. and van Loon, L. C. (1999). Salicylic acid-independent plant defence pathways. Trends Plant Sci. 4, 52-58.
Reinhart, B. J., Slack, F. J., Basson, M., Pasquinelli, A. E., Bettinger, J. C., Rougvie, A. E., Horvitz, H. R., and Ruvkun, G. (2000). The 21-nucleotide let-7 RNA regulates developmental timing in Caenorhabditis elegans. Nature 403, 901-906.
Ryals, J. A., Neuenschwander, U. H., Willits, M. G., Molina, A., Steiner, H. Y., and Hunt, M. D. (1996). Systemic acquired resistance. Plant Cell 8, 1809-1819.
Sambrook, J., Fritsch, E. F., and Maniatis, T. (1989). Molecular cloning. A laboratory manual. (New York: Cold Spring Harbor Laboratory Press).
Schweizer, P., Pokorny, J., Schulze, Lefert, P., and Dudler, R. (2000). Double-stranded RNA interferes with gene function at the single-cell level in cereals. Plant J. 24, 895-903.
Sharp, P. A. (2001). RNA interference 2001. Genes Devel. 15, 485-490.
Sheen, J., Hwang, S., Niwa, Y., Kobayashi, H., and Galbraith, D. W. (1995). Green-fluorescent protein as a new vital marker in plant cells. Plant J. 8, 777-784.
Sijen, T., Vijn, I., Rebocho, A., van Blokland, R., Roelofs, D., Mol, J. N. M., and Kooter, J. M. (2001). Transcriptional and posttranscriptional gene silencing are mechanistically linked. Curr. Biol. 11, 436-440.
Smith, N. A., Singh, S. P., Wang, M.-B., Stoutjesdijk, P. A., Green, A. G., and Waterhouse, P. M. (2000). Total silencing by intron spliced hairpin RNAs. Nature 407, 319-320.
Tuschl, T., Zamore, P. D., Lehmann, R., and Bartel, D. P. (1999). Targeted mRNA degradation by double stranded RNA in vitro. Genes Devel. 13, 3191-3197.
Vance, V. and Vaucheret, H. (2001). RNA silencing in plants: Defense and counterdefense. Science 292, 2277-2280.
Voinnet, O., Vain, P., Angell, S., and Baulcombe, D. C. (1998). Systemic spread of sequence-specific transgene RNA degradation in plants is initiated by localized introduction of ectopic promoterless DNA. Cell 95, 177-187.
Waterhouse, P. M., Graham, M. W., and Wang, M. B. (1998). Virus resistance and gene silencing in plants can be induced by simultaneous expression of sense and antisense RNA. Proc. Natl. Acad. Sci. USA 95, 13959-13964.
Waterhouse, P. M., Smith, N. A., and Wang, M. B. (1999). Virus resistance and gene silencing: killing the messenger. Trends Plant Sci. 4, 452-457.
Weinmann, P., Gossen, M., Hillen, W., Bujard, H., and Gatz, C. (1994). A chimeric transactivator allows tetracycline-responsive gene expression in whole plants. Plant J. 5, 559-570.
Zamore, P. D., Tuschl, T., Sharp, P. A., and Bartel, D. P. (2000). RNAi: Double-stranded RNA directs the ATP-dependent cleavage of mRNA at 21 to 23 nucleotide intervals. Cell 101, 25-33.

### SUMMARY OF THE INVENTION

The present invention addresses the need for methods to induce gene expression reproducibly and predictably in a cell. The present invention provides methods to regulate gene expression in eukaryotic cells, in particular by inducing stable and persistent expression of a desired gene. According to the present invention, a method is provided for inducing expression of a nucleic acid by providing a first nucleic acid comprising a sequence of interest operably linked to a repressible promoter; and decreasing the level of a repressor acting on the repressible promoter by using nucleic acid-mediated silencing of a second nucleic acid that controls expression of or encodes the repressor, to a level that allows expression of the first nucleic acid. In one embodiment the second nucleic acid controls expression of the repressor, whereas in an alternative embodiment the second nucleic acid encodes the repressor.

The repressible promoter can be one that is functional in a plant cell or a mammalian cell depending on the desired objective.

The nucleic acid-mediated silencing will typically take place in a cell and can be mediated by introducing additional copies of a transgene into the cell, in particular into a plant cell. Alternatively, the nucleic acid-mediated silencing can be mediated by single-stranded or double-stranded ribonucleic acids. The ribonucleic acid is typically at least 20 nucleotides in length, at least 50, at least 100, at least 300 nucleotides in length or longer.

The repressor may be selected by the practitioner to obtain the desired goal, such as using repressors functional in mammalian systems to attain induction of gene expression in mammalian cells. The repressor may be selected from the group consisting of tetracycline repressor, the lad repressor, Catharanthus roseus G-box binding factors 1 and 2, Drosophila Groucho or Kruepple, KAP-1, NcoR, SMRT, retinoblastoma proteins and KRAB domain proteins.

The first nucleic acid may be any nucleic acid, which it is desired to induce expression, including agriculturally relevant genes in crops, genes preferably obtained or derived from plants, or therapeutics in mammalian cells. The first nucleic acid can encode a sequence endogenous to the cell or exogenous to the cell, such as markers useful as research tools for identifying plants or cells exhibiting silencing or for screening mutants.

In another aspect of the invention, the method further comprises providing the first nucleic acid in a cell; providing an inducible promoter operably linked to the coding sequence of the repressor in the cell; providing a down-regulatable promoter operably linked to the coding sequence of the repressor in the cell; and administering an effective amount of inducer to allow expression of the repressor. The inducer can be, for example, a chemical, a pathogen infection or, in particular for plant cells, the inducer can be selected from the group consisting of heat, light, cold stress, UV light and ozone. In a further aspect, the method comprises administering an inhibitor that down-regulates the down-regulatable promoter. Alternatively, a tissue specific promoter could be used to achieve the ability to switch on or off the expression of the first nucleic acid.

In a further embodiment, a plant cell comprising a TET^{R} repressible promoter operably linked to a β-glucuronidase gene is provided, as well as a plant comprising the plant cell, progeny or seeds thereof.

### FIGURE LEGENDS

FIG. 1. Cartoons illustrating how expression of a TET^{R} repressible TX-GUS reporter gene (A) can be restored by treatment with anhydrotetracycline (aTc) (B), and by silencing of a 35S-TET^{R} transgene. Elliptical symbols represent proteins. Representative examples of untreated (D) and 15 μg/ml aTc-treated (E) Nt TET^{R}GUS plants stained for GUS 2 days later.

FIG. 2. (A) The incidence of GUS expression in a Nt TET^{R}GUS transformant bombarded with 35S TET^{R} plasmids and RNA molecules representing the TET^{R} transcribed region. Plantlets (12 days old with one true leaf) were bombarded with 5-10 μg of nucleic acid and stained for GUS ca. 12 days later. The incidence of GUS expression is shown as % plants showing blue GUS staining obtained in at least 3 independent experiments for the number of plants indicated in parenthesis. The sense- and antisense-smRNAs represent positions 517-537 and 535-514 of the transcribed region, respectively. Double stranded smRNA was obtained by spontaneous annealing of the single-stranded smRNAs at room temperature. The promoter (open bar), 3′- and 5′-UTR (cross hatched bars), coding region (solid bar with arrows showing orientation) are indicated for the DNA constructs. The length and orientation (solid arrows) of RNA molecules are indicated. Positions are relative to 5′-end of the RNA. (B) The incidence of silencing of 35S-GFP in a Nb GFP transformant bombarded with 35S-GFP plasmids and RNA molecules representing the GFP transcribed region. The conditions are the same as in (A). The sense- and antisense-smRNAs represent positions 556-576 and 574-553 of the transcribed region, respectively. The double-stranded smRNA with mismatches at 6 of 19 positions represents a transcribed region of a related sGFP gene (Sheen et al., 1995).

### DETAILED DESCRIPTION OF THE INVENTION

Posttranscriptional gene silencing (PTGS) in plants is an epigenetic form of RNA degradation with mechanistic and genetic links to PTGS and RNA interference (RNAi) in fungi and animals. The present inventors have developed a system for inducing gene expression based on the silencing of a repressor. Large sense, antisense, and double-stranded RNAs as well as shorted, 21-22 nucleotide-long, double-stranded RNAs (smRNA) delivered into cells are shown in the Examples below to induce expression of a desired gene in a manner that allows the spreading of expression from cell to cell and, in plants, systemic spreading. Thus, self-sustaining production of smRNAs is shown to be sufficient to maintain expression of the gene of interest.

In its broadest aspect, the present invention therefore provides a method of inducing expression of a nucleic acid by providing a first nucleic acid comprising a sequence of interest operably linked to a repressible promoter; and decreasing the level of a repressor acting on the repressible promoter by using nucleic acid-mediated silencing of a second nucleic acid to a level that allows expression of the first nucleic acid. The second nucleic acid can control expression of the repressor (such as, being a regulatory region affecting expression of the repressor) or can encode the repressor.

By “induced expression” it is meant that an increase in the amount of a product of gene expression (RNA or protein) is seen, which is larger than the margin of error inherent in the measurement technique, preferably an increase by about 2-fold or greater of the expression in a control cell, more preferably an increase by about 5-fold or greater, and most preferably an increase by about 10-fold or greater is seen. “Expression” refers to the transcription and/or translation of a nucleotide sequence, for example an endogenous gene or a heterologous gene, in a cell. Expression may therefore refer to transcription only.

The first nucleic acid may be any nucleic acid, for which it is desired to induce expression. The first nucleic acid can encode a sequence heterologous to the cell, such as markers useful as research tools for identifying plants or cells exhibiting silencing or for screening mutants. A heterologous DNA Sequence is a sequence not naturally associated with a host cell into which it is introduced. Choices for a non-endogenous (heterologous) marker gene include without limitation luciferase, green fluorescent protein (GFP), or beta-glucuronidase (GUS). Assay methods for each of these markers have been described (Ishitani et al. (1997) Plant Cell, 9:1935-1949; Cutler et al. (2000) Proc. Natl. Acad. Sci. USA 97: 3718-3723; Jefferson et al. (1989) EMBO J., 6:3901-3907).

Alternatively, the first nucleic acid may include agriculturally relevant genes in crops. Such genes are preferably obtained or derived from a plant, preferably from a monocotyledonous plant or a dicotyledonous plant. Preferably, the plants include, without limitation, corn, rice, wheat, soybean, cotton, sunflower, Brassica spp., canola, tomato, potato, Solanaceae spp. or sugar beets. A heterologous nucleotide sequence encodes for example, but not limited to, a polypeptide involved in waxy starch, herbicide tolerance, resistance for bacterial, fungal, or viral disease, insect resistance, enhanced nutritional quality, improved performance in an industrial process, altered reproductive capability, such as male sterility or male fertility, yield stability and yield enhancement. Using the present invention, such traits are reproducibly expressed in a plant cell during the life of the plant. Examples of endogenous nucleotide sequences of interest whose expression in a plant cell is altered using the present invention are found for example in WO 99/53050. An “endogenous” nucleotide sequence refers to a nucleotide sequence that is present in the genome of the cell.

In yet another embodiment, the first nucleic acid may include a therapeutic useful in treating disorders or diseases of mammalian cells, in particular human cells. Any desired product can be induced in this was and may include endogenous gene products, such as growth factors, hormones, erythropoietin, insulin, or immunoactive proteins, such as antibodies or fragments thereof, or heterologous gene products, such as anti-sense RNA, therapeutic peptides and the like.

The first nucleic acid containing a sequence of interest is operably linked to a repressible promoter. A “repressible promoter” is a promoter that is inhibited until released from a repressor function. Typically, this would be the binding of a repressor to the respective repressible promoter. A promoter comprises those sequences typically 5′ to any coding sequences necessary to allow expression of the transcript product. These may include a TATA box and various other regulatory regions as is known in the art. The repressible promoter should be chosen to be functional in the host cell of interest, for example, in a plant cell or a mammalian cell depending on the desired objective.

As described above, nucleic acid-mediated silencing has been observed under various conditions. The nucleic acid-mediated silencing will typically take place in a cell and can be mediated by introducing additional copies of a transgene into the cell, in particular into a plant cell. Alternatively, the nucleic acid-mediated silencing can be mediated by a single-stranded or double-stranded ribonucleic acid. A “double-stranded RNA” (dsRNA) comprises a sense RNA fragment of a nucleotide sequence and an antisense RNA fragment of the nucleotide sequence (e.g., repressor sequence), which both comprise nucleotide sequences complementary to one another, thereby allowing the sense and antisense RNA fragments to pair and form a double-stranded RNA molecule. The ds RNA can optionally comprise an overhang, as exemplified in the Examples below. The sequence of the dsRNA can be essentially the same as the promoter sequences (thus, inducing transcriptional gene silencing) or essentially the same as the coding or transcript region (inducing PTGS). Preferably, the sequence is chosen to be identical to the coding or transcript region. The ribonucleic acid is typically at least 20 nucleotides in length, preferably at least 50, more preferably at least 100, most preferably at least 300 nucleotides in length or longer.

The second nucleic acid can control expression of the repressor (such as, by interfering with a regulatory region affecting expression of the repressor) or can encode the repressor.

Preferably, the second nucleic acid encodes the repressor. The repressor—a negative regulator of gene expression—may be selected by the practitioner to obtain the desired goal, such as using repressors functional in mammalian systems to attain induction of gene expression in mammalian cells and those functional in plant systems to attain induction of gene expression in plant cells. The repressor may be any known repressor that functions in the target cell, for example, a repressor selected from the group consisting of tetracycline repressor, the lad repressor, Catharanthus roseus G-box binding factors 1 and 2, Drosophila Groucho or Kruepple, KAP-1, NCoR or SMRT (both of which have a negative regulatory effect on a steroid hormone receptor, e.g., estrogen receptor), specific histone deacetylases, retinoblastoma proteins (optionally complexed to E2F) and KRAB domain proteins, or fusions thereof.

In another aspect of the invention, the method further comprises providing the first nucleic acid in a cell; providing an inducible promoter operably linked to the coding sequence of the repressor in the cell; providing a down-regulatable promoter operably linked to the coding sequence of the repressor in the cell; and administering an effective amount of inducer to allow expression of the repressor. The inducer can be, for example, a chemical, such as salicylic acid or Bion TM, a pathogen infection or, in particular for plant cells, the inducer can be selected from the group consisting of heat, light, cold stress, UV light and ozone. Transcription of the repressor in the presence of inducer results in silencing of both nucleic acids comprising the repressor sequences, resulting in expression of the sequence of interest (first nucleic acid). The sequence of interest is preferably present in the genome of the plant cell but may be present in the plant cell as an extrachromosomal molecule. The advantage of this system over conventional induction with chemicals, for example, is that only a very small amount of inducer is needed to initiate induction of gene expression, which is then propagated from cell to cell without a requirement for the further presence of inducer.

In a further aspect, the method comprises administering an inhibitor that down-regulates the down-regulatable promoter. The inhibitor releases siliencing of the repressor gene expression, thereby resulting in expression on the target gene being switched off. An example of such a system would be the use of the class I β-1,3-glucanase promoter in plant cells, which is down-regulated by cytokinin, auxin and abscisic acid but up-regulated by ethylene. Alternatively, the inducer might be a pathogen and the inhibitor a drug, for example. The “switch” system allows the practitioner to regulate when expression of the target gene should occur using significantly lower quantities of inducer than previously used. For regulatable expression in plants, the chemically inducible PR-1 promoter from tobacco or Arabidopsis may be used (see, e.g., U.S. Pat. No. 5,689,044). In summary, the system allows not only for the induction of gene expression, but of stable gene expression, that is, gene expression that can persist over the lifetime of the organism.

In a further embodiment, a tissue specific promoter or pathogen-induced promoter, for example, is used to induce gene silencing and achieve the ability to switch on or off the expression of the first nucleic acid of interest. For example, selected promoters will express transgenes in specific cell types (such as, without limitation, neurons or adipocytes in animal systems, or leaf epidermal cells, mesophyll cells or root cortex cells in plant systems). Similarly, promoters can be selected for expression in specific tissues or organs (e.g., liver-specific, mammary gland-specific, prostate-specific or lymphoid-specific expression or, in plants, expression specific in roots, leaves or flowers, for example) The selection will reflect the desired location of accumulation of the gene product.

In another embodiment of the invention, a screening assay for compounds capable of inducing gene expression of the first nucleic acid can be envisioned. For example, the method described above can be used to discover new inducers or down-regulators affecting nucleic-acid mediated gene silencing and to identify genes regulating gene silencing. A chemical is applied to the host cell, such as transgenic plant, plant tissue, plant seeds or plant cells and to control cells and expression of the first nucleic acid of interest is determined after application of the chemical and compared.

To carry out the methods of the invention, nucleic acids will have been introduced into host cells. Methods of introducing nucleic acids into host cells, as well as producing cell lines, plant lines or transgenic animals, are well known to those of skill in the art. Ribonucleic acids that are introduced into cells may include naturally occurring or synthetic or artificial nucleotides. Such modified nucleic acids may be more resistant to degradation and can be used advantageously in the methods of the invention.

The present invention may also make use of plasmids, expression cassettes and viral vectors comprising a promoter operably linked to a transcribable nucleic acid molecule and a terminator, optionally with an enhancer. “Expression cassette” as used herein means a DNA sequence capable of directing expression of a particular nucleotide sequence in an appropriate host cell, comprising a promoter functional in the host cell into which it will be introduced, operatively linked to the nucleotide sequence of interest which is operatively linked to termination signals. It also typically comprises sequences required for proper translation of the nucleotide sequence. Nucleotide sequences of the present invention can be introduced and/or incorporated in animal, plant or bacterial cells using conventional recombinant DNA technology.

In particular, the coding sequence of the selected gene can optionally be genetically engineered by altering the coding sequence for optimal expression in the species of interest as is well known in the art. Transformation techniques can include the use of Agrobacterium, viral infection, particle bombardment, calcium phosphate-, PEG- or liposome-mediated transfer, electroporation and microinjection depending on the host cell.

Eukaryotic hosts for screening methods will include yeast, Drosophila, C. elegans and other higher organisms. Mammalian hosts will include animals of veterinary importance, as well as mice, rats, rabbits, dogs, cats, cattle, pigs and humans. However, in mammalian cells, gene silencing methods that avoid activation of or destroy the PKR pathway are preferred, for example using smRNAs, increasing expression of dicer to promote formation of smRNAs from longer substrates or co-transfecting with RNAi specific for the components of the PKR pathway (e.g., PKR, RNAseL).

Administration to individuals or animals of pharmaceutical inducers or expression constructs useful in carrying out the invention may be accomplished orally or parenterally, including by inhalation. Methods of parenteral delivery include topical, intra-arterial (e.g. directly to the tumour), intramuscular, subcutaneous, intramedullary, intrathecal, intraventricular, intravenous, intraperitoneal, or intranasal administration. The compositions can contain suitable pharmaceutically acceptable carriers comprising excipients or stabilizers, for example. Further details on techniques for formulation and administration can be found in the latest edition of Remington's Pharmaceutical Sciences (Maack Publishing Co, Easton Pa.). The pharmaceutical compositions can be manufactured in substantial accordance with standard manufacturing procedures known in the art. Dosages can be determined empirically depending on the desired effect using routine procedures known In the art.

Although the methods of the invention can be carried out on any cell, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

In a further embodiment, a plant cell is provided comprising a repressible promoter, such as the TET^{R} repressible promoter, operably linked to a nucleic acid of interest, such as the marker gene, β-galactosidase, as well as a plant comprising the plant cell, progeny or seeds thereof. A “plant” refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units.

The invention will be further described by reference to the following detailed examples. These examples are provided for purposes of illustration only, and are not intended to be limiting unless otherwise specified.

### EXAMPLES

### Example 1

### Induction of Reporter Activity in Plant Cells by DNA-Mediated Gene Silencing

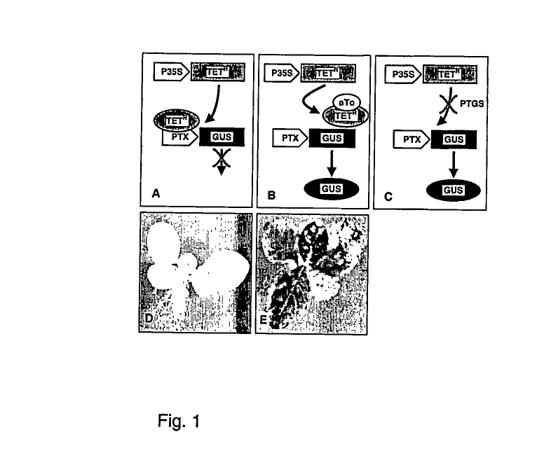

It has previously been reported (Voinnet et al., 1998; Palauqui and Balzergue, 1999) that gene silencing can be triggered by biolistic bombardment with additional copies of a resident transgene. The example below uses the introduction of additional copies of a resident transgene into a plant cell, to result in the induction of β-glucuronidase activity. The inventors have developed a gene expression system based on the use of a transcriptional repressor. Tobacco plants were transformed sequentially with a chimeric gene (35S-TET^{R}) encoding a bacterial tetracycline repressor (TET^{R}) regulated by the cauliflower mosaic virus 35S RNA promoter (Jones et al., 1998), and then with a chimeric E. coli β-glucuronidase (GUS) reporter gene regulated by the TET^{R} repressible TX promoter (Weinmann et al., 1994). FIG. 1A to C illustrates the principle of the assay. If TET^{R} is highly expressed in these Nt TET^{R} GUS transformants, then transcription of the TET^{R} repressible target gene will be blocked and no GUS should be detected by histological staining. In contrast, if expression of the TET^{R} gene is silenced, then the TET^{R} repressible reporter gene will be transcribed, GUS will accumulate, and the cells expressing GUS will exhibit a blue coloration.

Materials and Methods

Transgenic Plants: The line Nt TET^{R}GUS was obtained by Agrobacterium-mediated leaf-disc transformation (Horsch et al., 1988) of homozygous tobacco line R7 containing a 35S-TET^{R} transgene and hygromycin-resistance marker (Jones et al., 1998) with the plasmid pTX-GUS carrying a GUS reporter gene regulated by a TET^{R} repressible TX promoter and a kanamycin-resistance marker (Weinmann et al., 1994). Plants homozygous for a single TX-GUS T-DNA locus were obtained by selfing primary regenerates and selecting for kanamycin-resistant progeny. The Nb GFP line of N. benthamiana (control) carrying a mGFP-ER reporter gene with a 35S promoter and Nos terminator has been described (Voinnet et al., 1998). Plants were raised from seed in 10-cm diameter Petri dishes containing agar-solidified Linsmaier and Skoog medium (Linsmaier and Skoog, 1965) at 280 in constant light (3000 Lux), and then grown on soil in a phytotron at 25° (16 h 12,000 Lux light/8 hr dark). Cells with GUS activity were detected by histological staining (Klahre and Chua, 1999). To confirm that GUS expression is under the control of TET^{R}, three-week old, hydroponically grown, Nt TET^{R}GUS plants were fed via roots with 15 μg/ml anhydrotetracycline (Jones et al., 1998) for two days to inactivate the TET^{R} and then stained for GUS. Low-background Nt TET^{R} GUS transformants that showed substantial GUS activity only after treatment with anhydrotetracycline (FIGS. 1B, D and E) were chosen for further experiments to validate the assay system.

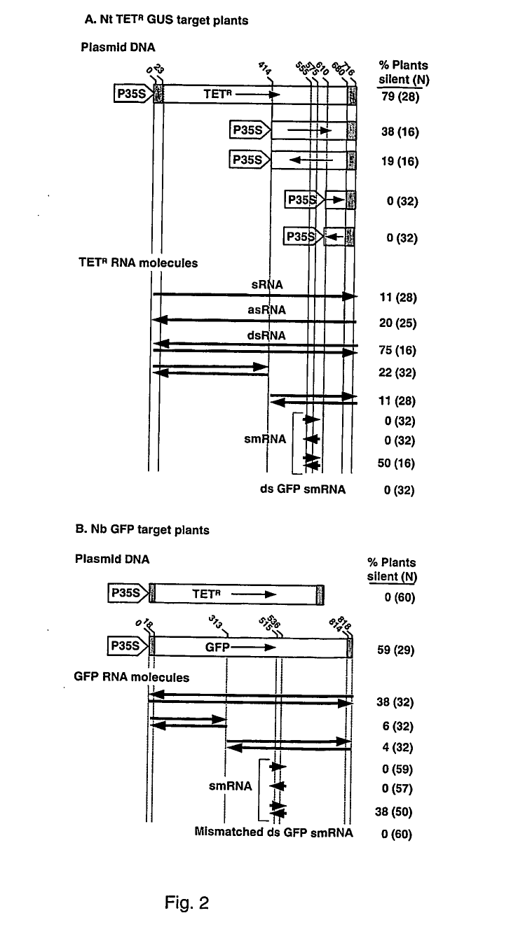

Plasmid Constructs: Plasmids used for biolistic experiments were prepared by standard methods (Sambrook et al., 1989). Plasmid p35S-TET^{R} is the EcoRI-HindIII fragment of pTET1 (Gatz et al., 1992) containing TET^{R} with a 35S promoter and ocs terminator cloned into pBS KS (Stratagene) cut with EcoRI and HindIII. The truncations p35S-TET^{R}₀₋₄₁₄, p35S-TET^{R}₄₁₄₋₇₁₆, and p35S-TET^{R}₆₁₀₋₇₁₆ contain the BamHI-NsiI and BamHI-NdeI fragments respectively of p35S-TET^{R} in sense and antisense orientation as indicated in FIG. 2A. Plasmid p35S-GFP is pUC18 containing mGFP-ER with a 35S promoter and Nos terminator (Haseloff et al., 1997). The truncations p35S-GFP₀₋₃₁₃ and p35S-GFP₃₁₃₋₈₁₈ contain the BamHI-NdeI and NdeI-SacI fragments, respectively, of the mGFP-ER transcribed region used for in vitro transcription.

Biolistic Bombardment and Detection of Silencing: Axenically grown plants, 12 days (7-10 days for GFP plants) after sowing and with one true leaf, were bombarded using a biolistic PDS-1000/He particle gun (BioRad, Richmond, Calif.). The plasmids 35S-TET^{R} or p35S-GFP were loaded on gold particles and were delivered to the appropriate plant at 1100 psi following the manufacturer's recommendations. Silencing of TET^{R} and GFP transgenes was detected, respectively, by histological staining of GUS and by visual inspection of plants illuminated with a 100 W “blue light” lamp Model B100-AP (UVP, Upland, Calif., USA). Images were collected with a Powershot Pro 70 digital camera (Canon, Japan) and a Leica DMRD microscope (Heidelberg, Germany) equipped with a cooled CCD camera and SPOT 3.0.4 software (Diagnostic Instruments, Sterling Heights, Mich., USA).

The efficiency of different constructs to trigger silencing was expressed as the percentage of bombarded plants showing blue regions indicative of GUS activity on the bombarded leaves or elsewhere on the plant. Under the conditions used, blue coloration precisely reflects cells with GUS activity (Iglesias et al., 1994). FIG. 2A shows that ca. 79% of plants bombarded with plasmid DNA carrying the full-length 35S-TET^{R} gene exhibited regions of blue coloration. A lower incidence of GUS expression was detected with constructs containing ca. 300 bp of 3′-transcribed region in sense as well as in antisense orientation. GUS expression was not detected with gold particles without DNA or with constructs containing ca. 100 bp of 3′-transcribed region in either orientation. These results show that efficiency decreases with size of the transcribed region; and, that genes transcribed in both orientations are effective. In addition, the results show that gene silencing can be used to induce gene expression, as exemplified by the TET^{R} GUS system.

To rule out the possibility that the effects observed were a special feature of the TET^{R} GUS system or plant species used, silencing activity of a plasmid expressing an unrelated green fluorescent protein (GFP) reporter gene can be determined in Nicotiana benthamiana transformed with a chimeric 35S-GFP gene (Nb GFP)(Voinnet et al., 1998). Plants are viewed under blue light and scored for regions showing red autofluorescence of chlorophyll indicative of silencing, which is masked by green fluorescence due to GFP in GFP expressing tissues. Additional copies of 35S-GFP DNA introduced biolistically into Nb GFP 7-10 days after germination triggered silencing in a sequence-specific fashion (FIG. 2B).

### Example 2

### Induction of Reporter Activity in Plant Cells by RNA-Mediated Gene Silencing

The ability of high-molecular weight RNA to trigger silencing was tested by biolistic delivery of DNase-treated RNA preparations obtained by in vitro transcription. Unless otherwise stated or clear from the context, the methodology was essentially as described in Example 1. RNA transcripts were produced with the relevant fragments of the transcribed regions (see FIG. 2) of TET^{R} and mGFP-ER cloned into pBS-SK- as templates and treated with DNase using a “Megascript” transcription kit (Ambion, Austin, Tex., USA) according to the manufacturer's recommendations. Typical yields were 50 μg RNA using 1 μg of DNA template. Integrity of the transcripts was verified by agarose-gel electrophoresis under denaturing conditions. RNA transcripts were annealed by heating at 95° for 2 minutes, and slowly cooling to 37° over a period of 5 minutes. To test for RNase sensitivity, single-stranded or annealed RNA was precipitated with ethanol and then incubated in 40 μg/ml RNase A, 200 mM NaCl, 100 mM LiCl, 1 mM EDTA, 10 mM Tris buffer, pH 7.5 for 30 minutes at 250.

Double stranded RNA (dsRNA) representing the entire transcribed region of the TET^{R} gene gave a high ca. 75% incidence of GUS expression (FIG. 2A). Lower efficiencies were obtained with shorter 414- and 303-ntd long dsRNAs representing the 5′- and 3′-ends of the transcribed region. Table 1 shows that comparable doses of dsRNA and plasmid DNA expressed on a μg basis give approximately the same incidence of GUS expression. Together with the high, 50-fold yield of RNA product relative to DNA template obtained by in vitro transcription and the fact that RNA preparations were treated with DNase, it seems unlikely that the silencing activity of the RNA preparations is due to traces of DNA. Full-length sense and antisense RNAs also resulted in GUS activity, but at lower efficiencies than those obtained with dsRNA (FIG. 2A). The effect of single-stranded RNA, but not that of dsRNA was abolished by incubating the preparations with RNase A supporting the conclusion that single-stranded RNA can induce GUS expression and that GUS expression obtained with dsRNA preparations is not due to contamination with the single-stranded RNAs.

As described in Example 1 for DNA, our control data confirmed the silencing activity of RNAs for an unrelated green fluorescent protein (GFP) reporter gene in Nicotiana benthamlana transformed with a chimeric 35S-GFP gene (Nb GFP)(Voinnet et al., 1998) (see FIG. 2B).

### Example 3

### Induction of Reporter Activity in Plant Cells by Short Oligoribonucleotide-Mediated Gene Silencing

Reports that double-stranded smRNAs could trigger degradation of target RNAs in Drosophila extracts (Zamore et al., 2000; Elbashir et al., 2001) and RNAi when introduced into cultured mammalian cells (Elbashir et al., 2001) prompted us to examine the possibility that these oligo-ribonucleotides might also effectively induce gene expression in our system. We tested chemically synthesized 21-nucleotide sense TET^{R} smRNA, 22-nucleotide antisense TET^{R} smRNA, and double-stranded smRNA with 2- and 3-nucleotide 3′-overhangs essentially as described in Example 1.

Oligo-ribonucleotides (smRNAs) representing regions of TET^{R} (Gatz et al., 1992), mGFP-ER (Haseloff et al., 1997), and sGFP (Sheen et al., 1995) transcripts were purchased from Mycrosynth (Balgach, Switzerland). Positions of 5′- and 3′-ends relative to the 5′-end of the transcripts are indicated in parentheses:

| | |
|---|---|
| Sense TET^{R} smRNA: | |
| 5′(517)-UGAUAGUAUGCCGCCAUUAUU-3′(537) | SEQ ID NO:1 |
| Antisense TET^{R} smRNA: | |
| 5′(535)-UAAUGGCGGCAUACUAUCACUA-3′(514) | SEQ ID NO:2 |
| Sense mGFP-ER smRNA: | |
| 5′(556)-AGAACGGCAUCAAAGCCAACU-3′(576) | SEQ ID NO:3 |
| Antisense mGFP-ER smRNA: | |
| 5′(574)-UUGGCUUUGAUGCCGUUCUUUU-3′(553) | SEQ ID NO:4 |
| Sense sGFP smRNA: | |
| 5′(193)-UUCACCUACGGCGUGCAGUGC-3′(213) | SEQ ID NO:5 |
| Antisense sGFP smRNA: | |
| 5′(211)-ACUGCACGCCGUAGGUGAAGGU-3′(190) | SEQ ID NO:6 |

Double-stranded smRNAs with 2- and 3-nucleotide 3′-overhangs were obtained by spontaneous annealing of mixtures of the antisense and sense oligo-ribonucleotides at room temperature. Sense and antisense oligo-deoxyribonucleotides representing the oligo-nucleotide sequences described above were chemically synthesized and annealed to give double-stranded oligomers.

Double-stranded TET^{R} smRNA induced substantial GUS expression (FIG. 2A). We found that double-stranded smRNAs were roughly as efficient as dsRNA (Example 2) on a mass basis, but ca. 30-fold less effective on a molar basis (see Table 1). No induction was detected with single-stranded TET^{R} smRNAs in either orientation or with a double-stranded smRNA of the same length but unrelated in sequence. As described in Example 1 for DNA and Example 2 for longer RNA transcripts, our control data confirmed the silencing activity of smRNAs for an unrelated green fluorescent protein (GFP) reporter gene in Nicotiana benthamiana transformed with a chimeric 35S-GFP gene (Nb GFP)(Voinnet et al., 1998) (see FIG. 2B). No silencing was observed using a double-stranded GFP smRNA with mismatches at 6 of 19 positions indicating that silencing triggered by smRNAs is highly sequence specific.

To determine if oligo-deoxyribonucleotides could induce GUS activity, we tested sense-, antisense-, and double stranded-oligodeoxyribonucleotides equivalent in sequence to the smRNAs shown earlier to be effective. In no case was silencing observed.

### Example 4

### Gene Expression does not Remain Localized But Spreads from Cell-to-Cell and Systemically

The cell-to-cell spread of plasmid DNA- and RNA-induced GUS expression described in Examples 1 and 2 were monitored, as agricultural and pharmaceutical applications are potentially dependent thereon. Experiments were carried out essentially as described in the preceeding Examples. Twelve days after bombardment of Nt TET^{R}GUS plants with 35S-TET^{R} DNA, high-molecular weight single- and double-stranded TET^{R} RNAs, and double-stranded TET^{R} smRNAs GUS staining was confined to the bombarded leaf. Initially, staining was detected in groups of epidermal cells including trichomes and guard cells, but never in single epidermal cells. Later, staining appeared to spread to small groups of mesophyll cells, which were sometimes adjacent to small, densely stained veins. This indicates that using our system GUS expression in the bombarded leaf spread locally from cell to cell and possibly via small veins as well.

GUS staining of entire plants one month after bombardment revealed that staining resulting from the effect of 35S-TET^{R} DNA, single- and double-stranded TET^{R} RNA and double-stranded TET^{R} smRNA can spread systemically to the veins of non-bombarded leaves. We found that single-stranded, high-molecular weight transcripts generated by in vitro transcription can trigger systemic effects, but at a lower efficiency than dsRNAs.

Real-time monitoring of control Nb GFP plants showed that 35S-GFP DNA, double-stranded high molecular-weight RNA and GFP smRNA resulted in patches of silencing on bombarded leaves that gradually increased in size. Occasionally, silent regions were detected one day after bombardment. After 3-4 days the majority of bombarded leaves showed conspicuous regions of silencing. Systemic spread of silencing was evident in plants two weeks after bombardment (starting with the veins of nonbombarded leaves) and after 1 month was evident in nonvascular tissues.

In summary, all of the RNA species we tested were able to elicit production of a desired gene product with an expression pattern that is capable of spreading into surrounding cells and even systemically, with potential agricultural and pharmaceutical applications. Although the Examples above exemplify the invention with the TET repressor and GUS reporter activity, it will be clear to one of ordinary skill in the art these can be easily modified to use other repressor systems or to obtain other gene products. Our results with the TET^{R} system therefore only illustrate how silencing of repressors might serve as a mechanism for stable activation of gene expression.

### Example 5

### Double-Stranded siRNA Induces PTGS and the Accumulation of Newly Formed siRNA in Nonbombarded Leaves

RNA-blot hybridization was used to compare the accumulation of GFP mRNAs in highly GFP-expressing leaves of Nb GFP plants and in completely silenced nonbombarded leaves of Nb GFP plants bombarded with 35S-GFP plasmid DNA, high molecular weight GFP dsRNA, and double-stranded GFP siRNA. Leaves were harvested for RNA isolation 1 month after bombardment. Leaves were chosen that were not present at the time of the bombardment, but were completely silent as judged from the absence of GFP fluorescence (Silent) or from control plants showing high GFP fluorescence (High). Silencing of systemic leaves was correlated with a dramatic decrease in GFP mRNA accumulation.

The leaves were also assayed for siRNAs, which are a hallmark of silencing. Fractions enriched for small RNAs were hybridized with DNA probes representing the 3′ and 5′ regions of GFP mRNA. Small interfering (si)RNAs approximately 21 and 23 nt in length representing both regions of GFP mRNA accumulated in systemically silent leaves ob-tained by bombardment with plasmid DNA, dsRNA, and double-stranded siRNA, but not in highly expressing leaves. Together, these results confirm that the RNAs tested induce systemic silencing at the posttranscriptional level. The 3′- and 5′-probes used for RNA-blot hybridization do not include the region of GFP mRNA identical in sequence to the siRNA used to induce siliencing. This fact indicates that biolistically delivered siRNA induces the de novo formation of siRNAs that accumulate in systemically silenced tissues.

The biolistic approach offers several advantages, such as, the potential effects of viral RNA replication, expression of viral RNA-dependent RNA polymerases (RdRPs), transcription of delivered DNA, or the delivered DNA itself are excluded.

The present invention is not to be limited in scope by the specific embodiments described which are intended as single illustrations of individual aspects of the invention, and any constructs, nucleic acid sequences or transformed plants which are functionally equivalent are within the scope of this invention. Indeed, various modifications of the invention in addition to those shown herein will become apparent to those skilled in the art from the foregoing description and accompanying drawings. Such modifications are intended to fall within the scope of the appended claims.

The present invention is not to be limited in scope by the specific embodiments described which are intended as single illustrations of individual aspects of the invention, and any constructs, nucleic acid sequences or transformed plants which are functionally equivalent are within the scope of this invention. Indeed, various modifications of the invention in addition to those shown herein will become apparent to those skilled in the art from the foregoing description and accompanying drawings. Such modifications are intended to fall within the scope of the appended claims.

Various patents and references are cited within the present specification, all of which are incorporated by reference in their entireties.

TABLE 1
The Effect of Plasmid and RNA Dose on the Incidence of Silencing

| | | Amount loaded | | % Silent |
|---|---|---|---|---|
| Target plant | Material delivered | μg | fmol | plants (N)^{a} |
| Nt TET^{R}GUS | p35S-TET^{R} DNA | 0.04 | 15. | 0 (24) |
| | | 0.2 | 77. | 32 (25) |
| | | 1.0 | 384. | 70 (17) |
| | | 10. | 3.9 × 10³ | 86 (15) |
| | TET^{R} dsRNA | 0.008 | 18. | 0 (19) |
| | | 0.04 | 93. | 6 (17) |
| | | 0.2 | 466. | 36 (14) |
| | | 1.0 | 2.33 × 10³ | 64 (14) |
| | | 15. | 3.49 × 10⁴ | 75 (15) |
| | TET^{R} 23-ntd | 0.1 | 7.75 × 10³ | 0 (24) |
| | ds smRNA | | | |
| | | 1.0 | 7.75 × 10⁴ | 13 (24) |
| | | 10. | 7.75 × 10⁵ | 43 (30) |
| Nb GFP | p35S-GFP DNA | 0.04 | 15. | 0 (32) |
| | | 0.2 | 75. | 9 (32) |
| | | 1.0 | 373. | 38 (32) |
| | | 5.0 | 1.86 × 10³ | 56 (32) |
| | GFP dsRNA | 0.04 | 81. | 0 (32) |
| | | 0.16 | 326. | 28 (32) |
| | | 0.8 | 1.63 × 10³ | 28 (32) |
| | | 4.0 | 8.15 × 10³ | 50 (32) |
| | | 20. | 4.08 × 10⁴ | 38 (32) |
| | GFP 23-ntd ds | 0.15 | 1.16 × 10⁴ | 0 (15) |
| | smRNA | | | |
| | | 1.5 | 1.16 × 10⁵ | 13 (16) |
| | | 15. | 1.16 × 10⁶ | 50 (16) |

^{a}Nucleic acids were delivered biolistically and plants scored for incidence of silencing as described in FIG. 2.

1. A method of inducing expression of a nucleic acid, said method comprising:
a. providing a first nucleic acid comprising a sequence of interest operably linked to a repressible promoter; and
b. decreasing the level of a repressor acting on said repressible promoter by using nucleic acid-mediated silencing of a second nucleic acid, said second nucleic acid controlling expression of or encoding said repressor, to a level that allows expression of said first nucleic acid.

2. The method of claim 1, wherein said second nucleic acid controls expression of said repressor.

3. The method of claim 1, wherein said second nucleic acid encodes said repressor.

4. The method of claim 1, wherein said repressible promoter is functional in a plant cell.

5. The method of claim 1, wherein said repressible promoter is functional in a mammalian cell.

6. The method of claim 1, wherein said nucleic acid-mediated gene silencing takes place in a cell and is mediated by introducing additional copies of a transgene into said cell.

7. The method of claim 1, wherein said nucleic acid-mediated gene silencing is mediated by double-stranded ribonucleic acid.

8. The method of claim 7, wherein said double-stranded ribonucleic acid is at least 20 nucleotides in length.

9. The method of claim 7, wherein said double-stranded ribonucleic acid is at least 300 nucleotides in length.

10. The method of claim 1, wherein said repressor is selected from the group consisting of tetracycline repressor, the lad repressor, Catharanthus roseus G-box binding factor 1, Catharanthus roseus G-box binding factor 2, Groucho, Kruepple, KAP-1, NcoR, SMRT, retinoblastoma protein and KRAB domain protein.

11. The method of claim 1, wherein said first nucleic acid encodes a polypeptide involved in waxy starch, herbicide tolerance, resistance for bacterial, fungal, or viral disease, insect resistance, enhanced nutritional quality, improved performance in an industrial process, altered reproductive capability, such as male sterility or male fertility, yield stability or yield enhancement.

12. The method according to claim 1, wherein said first nucleic acid is defined by a nucleotide sequence obtained or derived from a plant.

13. The method of claim 1, wherein said first nucleic acid encodes an endogenous nucleotide sequence of a target cell.

14. The method of claim 1, wherein said first nucleic acid is a therapeutic.

15. The method of claim 1, wherein said first nucleic acid is a marker.

16. The method of claim 1, said method further comprising:
providing said first nucleic acid in a cell;
providing an inducible promoter operably linked to the coding sequence of said repressor in said cell;
providing a down-regulatable promoter operably linked to the coding sequence of said repressor in said cell; and
administering an effective amount of inducer to allow expression of said repressor.

17. The method of claim 16, wherein said inducer is a chemical.

18. The method of claim 16, wherein said inducer is a pathogen infection.

19. The method of claim 16, wherein said cell is a plant cell and said inducer is selected from the group consisting of heat, light, cold stress, UV light and ozone.

20. The method of claim 16, further comprising administering an inhibitor that down-regulates said down-regulatable promoter.

21. A plant cell comprising a TET^{R} repressible promoter operably linked to β-glucuronidase gene.

22. A plant comprising the plant cell of claim 21, or progeny or seeds thereof.

23. The method according to claim 6, wherein said first nucleic acid is defined by a nucleotide sequence obtained or derived from a plant.

24. The method of claim 6, wherein said first nucleic acid encodes an endogenous nucleotide sequence of a target cell.

25. The method of claim 5, wherein said first nucleic acid is a therapeutic.


SEQUENCE LISTING (6 sequences)

SEQ ID NO: 1 - RNA, 21 nt, Artificial Sequence
  Description of Artificial Sequence oligonucleotide
ugauaguaugccgccauuauu

SEQ ID NO: 2 - RNA, 22 nt, Artificial Sequence
  Description of Artificial Sequence oligonucleotide
uaauggcggcauacuaucacua

SEQ ID NO: 3 - RNA, 21 nt, Artificial Sequence
  Description of Artificial Sequence oligonucleotide
agaacggcaucaaagccaacu

SEQ ID NO: 4 - RNA, 22 nt, Artificial Sequence
  Description of Artificial Sequence oligonuleotide
uuggcuuugaugccguucuuuu

SEQ ID NO: 5 - RNA, 21 nt, Artificial Sequence
  Description of Artificial Sequence oligonucleotide
uucaccuacggcgugcagugc

SEQ ID NO: 6 - RNA, 22 nt, Artificial Sequence
  Description of Artificial Sequence oligonucleotide
acugcacgccguaggugaaggu